(19) United States
(12) Patent Application Publication
(10) Pub. No.: US 2021/0306808 A1
(43) Pub. Date: Sep. 30, 2021

(54) SYSTEMS AND METHODS FOR DIGITAL CONTACT TRACING

(71) Applicant: Contakt LLC, Oceanside, CA (US)

(72) Inventor: Justin S. BECK, Oceanside, CA (US)

(21) Appl. No.: 17/219,302

(22) Filed: Mar. 31, 2021

Related U.S. Application Data
(60) Provisional application No. 63/100,755, filed on Mar. 31, 2020.
(60) Provisional application No. 63/083,776, filed on Sep. 25, 2020.
(60) Provisional application No. 63/111,857, filed on Nov. 10, 2020.
(60) Provisional application No. 63/111,861, filed on Nov. 10, 2020.

(51) Int. Cl. H04W 4/029; H04W 4/02; H04W 12/63

(52) U.S. Cl. CPC H04W 4/029; H04W 12/63; H04W 4/025; H04W 4/023

(57) ABSTRACT

A user system comprising: at least one hardware processor; and one or more software modules that are configured to, when executed by the at least one hardware processor, download an application to the user system; obtain an identifier for the user system; generate a plurality of pings to detect other nearby user systems; receive a response to one of the plurality of pings from another user system; exchange contact data with the responding user system; store the contact data for the responding user system; and receive a digital handshake indicated, based on the stored contact data that a user of the user system may be at risk.

### CROSS REFERENCE TO RELATED APPLICATIONS

This application claims priority to U.S. Provisional Application, 63/100,755, filed Mar. 31, 2020, entitled “Secure contact tracing software application running on mobile device nodes to execute blockchain contracts when entering physical or digital proximity, providing automatic, historical and predictive geolocation for mobile devices, and contacts,” and U.S. Provisional Application, 63/083,776, filed Sep. 25, 2020, entitled “SYSTEMS AND METHODS FOR DIGITAL CONTACT TRACING,” and U.S. Provisional Application, 63/111,857, filed Nov. 10, 2020, entitled “SYSTEMS AND METHODS FOR DIGITAL CONTACT TRACING USING RISK STRATIFICATION EVENTS,” and U.S. Provisional Application, 63/111,861, filed Nov. 10, 2020, entitled “SYSTEMS AND METHODS FOR DIGITAL CONTACT TRACING, FOLLOW UP AND INTERVENTIONS USING RISK STRATIFICATION EVENTS,” the contents of which are hereby incorporated by reference in their entirety.

### BACKGROUND

### 1. Technical Field

The embodiments described herein are related to automatic contact tracing, and in particular automatic contact tracing that uses risk stratification and built in work flows.

### 2. Related Art

Robust systems and methods for contact tracing are clearly needed to help combat a pandemic. Unfortunately, no such systems and methods currently exist. Attempts have been made to use data from people's mobile phones, via the Google and Apple API's, to track movement and contact with others. But this has proved difficult, because the data is decentralized, i.e., it is on the user's devices. As a result, the data is often not immediately available, and accessing it is highly invasive. Moreover, such tracing may be difficult for certain populations based on a lack of smartphones. Businesses, organizations, and physical locations in general also lack integrated solutions in which they can manage infection chains of contagions which may affect their staff, visitors, customers either arriving to or coming from their GPS location. Health agencies have no way to contextualize the data from certain populations and the sites that they visit, making pandemic response and contact tracing extremely fragmented, and lacking in the scale necessary to mitigate risk substantially.

Effective contact tracing is the most reliable way to mitigate any contagion, epidemic, or pandemic. Unfortunately, e.g., in the United States, contact tracing is a manual process that does not yet harness the power of available technologies. But as noted by the Center for Disease Control “Even one missed contact can keep the outbreak going.” Human lives and the global economy simply cannot count upon the fallibility of the human mind in such a crisis.

The purpose of contact tracing is to first identify people who have come in close contact with someone who is infected with a virus, such as the COVID-19 or Ebola virus. Such people are at higher risk of becoming infected themselves, and of potentially further infecting others. Closely watching these contacts after exposure to an infected person can help the contacts to get care and treatment, if required, which can prevent further transmission of the virus. This monitoring process is called contact tracing, which can be broken down into 3 basic steps: 1. Contact identification: Once someone is confirmed as infected with a virus, contacts are identified by asking about the person's activities and the activities and roles of the people around them since onset of illness. Contacts can be anyone who has been in contact with an infected person: family members, work colleagues, friends, or health care providers. 2. Contact listing: All persons considered to have contact with the infected person should be listed as contacts. Efforts should be made to identify every listed contact and to inform them of their contact status, what it means, the actions that will follow, and the importance of receiving early care if they develop symptoms. Contacts should also be provided with information about prevention of the disease. In some cases, quarantine or isolation is required for high risk contacts, either at home, or in hospital. 3. Contact follow-up: Regular follow-up should be conducted with all contacts to monitor for symptoms and test for signs of infection.

Contacts are watched for signs of illness for, e.g., 21 or 14 days from the last day they came in contact with the patient. If the contact develops a fever or other symptoms, they can be immediately isolated, tested, provided care, and the cycle starts again—all of the new patient's contacts are found and watched. But again, even one missed contact can keep the outbreak going.

Problems with current contact tracing include: Accessibility: Contact Data is not centralized for use by health professionals, resulting in unquantified inefficiencies. Mobile devices or human population as the case may be, can be hard to reach. Accuracy/reliability: Mitigation is not quantified or conducted according to reliable data. Upon reaching a human being in contact tracing, health professionals are reliant upon the accuracy of the information those individuals provide, including those shaped by any unknown motivations (fear for loved ones, malice, etc.). The information, by its nature, is not reliable. Intervention: Again, wasting precious moments to mitigate contagions are wasted on “manual” intervention that does not leverage modern technologies due to privacy issues or want of scalable solution. Intervention is achieved through considerable resources and bandwidth, on data known to be unreliable. Scale: As with global pandemics such as COVID-19, the scale of intervention is limited by time allocated by health professionals. Automation/intelligence: The entire healthcare system and related government response as we know it for contact tracing, is “read, react, hope” rather than “analyze, extrapolate, and execute.”

### SUMMARY

Systems and methods for digital contact tracing are described herein.

According to one aspect, a platform for digital contact tracing, comprising: a secure database comprising contact data for a plurality of users, associated with a plurality of mobile devices; at least one hardware processor; and one or more software modules that are configured to, when executed by the at least one hardware processor, assign a default risk level to each of the plurality of users, wherein patients under investigation from digital handshakes, or positive tests imported or integrated with the system, or any proxy for a positive test, i.e., information known to constitute a likely infection like a survey provided to a user of the system via app., chat, text, interactive voice recording, email, website, or form on a website, or that may be determined according to local protocols of a health agency are assigned a positive risk level, and wherein when digital handshakes record close contact between a user of the plurality of users and a positive user based on the contact data, then the user is assigned a high risk level, and wherein a user of the plurality of users is determined to be a close contact of a user assigned a high risk level based on close contact to a high risk user and based on the contact data, then the user is assigned a medium risk level, and wherein when a user of the plurality of users is determined to be a close contact of a user assigned a medium risk level based on close contact to a medium risk user and based on the contact data, then the user is assigned a medium risk level.

These and other features, aspects, and embodiments are described below in the section entitled “Detailed Description.”

### BRIEF DESCRIPTION OF THE DRAWINGS

Features, aspects, and embodiments are described in conjunction with the attached drawings, in which:

FIG. 1 is a diagram illustrating a digital contact tracing system and platform in accordance with one embodiment;

FIG. 2 is a diagram illustrating the installation of an application onto a mobile device that can perform and manage the device to device communication in accordance with one embodiment;

FIG. 3 is a diagram illustrating how the application and device of FIG. 2 can interface with a blockchain ledger in order to securely maintain the contact information in accordance with one example embodiment;

FIG. 4 is a diagram illustrating a ping radius for device initiated under the control of application in accordance with one example embodiment;

FIG. 5 is a diagram illustrating the sending of pings in accordance with one embodiment;

FIG. 6 is a diagram illustrating the sending of contact data between devices in accordance with one embodiment;

FIG. 7 is a diagram illustrating the storing of the contact data by each device in accordance with one embodiment;

FIG. 8 is a diagram illustrating an authorized user accessing the contact data in accordance with one embodiment;

FIG. 9 is a diagram illustrating an authorized user accessing a device in accordance with one embodiment;

FIG. 10 is a screenshot of a dashboard that can be used for managing contact tracing according to one embodiment;

FIG. 11 illustrates an example infrastructure, in which one or more of the processes described herein, may be implemented, according to an embodiment; and

FIG. 12 illustrates an example processing system, by which one or more of the processes described herein, may be executed, according to an embodiment.

### DETAILED DESCRIPTION

The disclosure and the various features and advantageous details thereof are explained more fully with reference to the non-limiting embodiments and examples that are described and/or illustrated in the accompanying drawings and detailed in the following. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure. Moreover, it is noted that like reference numerals represent similar parts throughout the several views of the drawings.

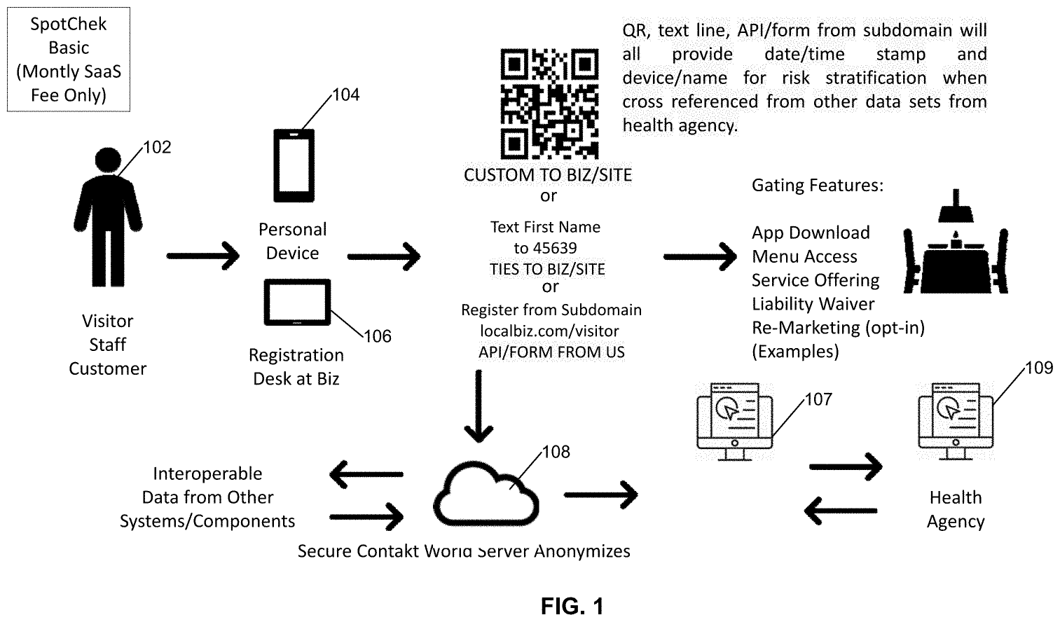

As illustrated in FIG. 1, a visitor 102 to a physical site will register their visit prior to entry so that their location and contacts can then be traced. There are several ways that visitor 102 can register. For example, visitor 102 can scan a QR or other type of code using their mobile device 104. Alternatively, or in the event visitor 102 cannot use their phone to scan, visitor 102 can send a text message to a dedicated text line. If visitor 102 cannot do either, then the user can simply provide their first name, last initial, email address, or other information which allows contact tracing associated with visitor 102's visit to a physical site at a certain date and time.

The code and/or text line information can be presented on device 106 or alternatively can be printed out for each visitor 102.

The QR code and text message lines can be provided by a web interface, custom to the location. In other words, a registration desk, table, kiosk, etc., can be set up with a computer or other device 106 that will present an interface that can present the code and/or text number to a visitor 102. Again, visitor 102 cannot scan or text, then they can provide relevant identifying information through the web interface on device 106.

The QR or other code 108 scan results in the optional download of a mobile application onto the visitor's mobile phone 104, if possible, which registers their visit to the site and records the time, date, mobile device identifier, and the QR Code. The visitor can also be required to provide personal details, such as their email address, through the application. If the application cannot be downloaded, then the user can register via the web interface on device 106.

If the user is texting, then the text message results in registration of the device as a visitor to the site, sends a confirmation message to the visitor 102 that they can enter the site (to show someone if needed), and records the time, date, and the mobile device identifier. The user may also be required to provide personal information either via text or the web interface.

The recorded information can be stored via a platform server (not shown) that can be remote from the site, e.g., in the cloud 108. The recorded information is of course tied to the entity or physical site, e.g., company or school that is controlling access to the site. The entity can then access this information via a portal and using a computer or device 110. Health agencies 112 can also access the data via a portal. Access to and security of the data can comply with HIPPA and other privacy or healthcare regulations. For example, the data can be de-identified and protected against unauthorized access.

The registration can be used to not only control access but to also gate access to services offered. For example, if the site is a restaurant, registration may need to be completed before the visitor 102 can access a menu. The entity or company managing the site may require a waiver before access to the site and/or services is provided. The waiver can be administered through the mobile application or device 106.

Moreover, the mobile application can be associated with the site or entity and present an opportunity for marketing to and staying connected with the visitor 102. Of course, this may be at the option of the visitor 102.

But more importantly, if an infection chain for COVID-19 or any other contagious disease is identified from the site or to the site, either the business itself or the local health agency can contact all related devices in real time from using data presented via the respective portals. In this way, effective, timely, and easy contact tracing can take place.

In certain embodiments, the reporting and tracing for people entering or associated with a venue can be used to generate a risk score for the venue. The score can take many forms, e.g., a scale from 1-100. In this manner, the venue owner/operator can assess the risk associated with their venue or health officials/agencies can make decisions without violating anyone's personal privacy or violating any privacy/security laws or regulations.

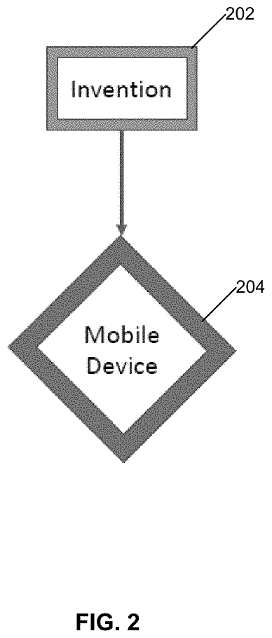

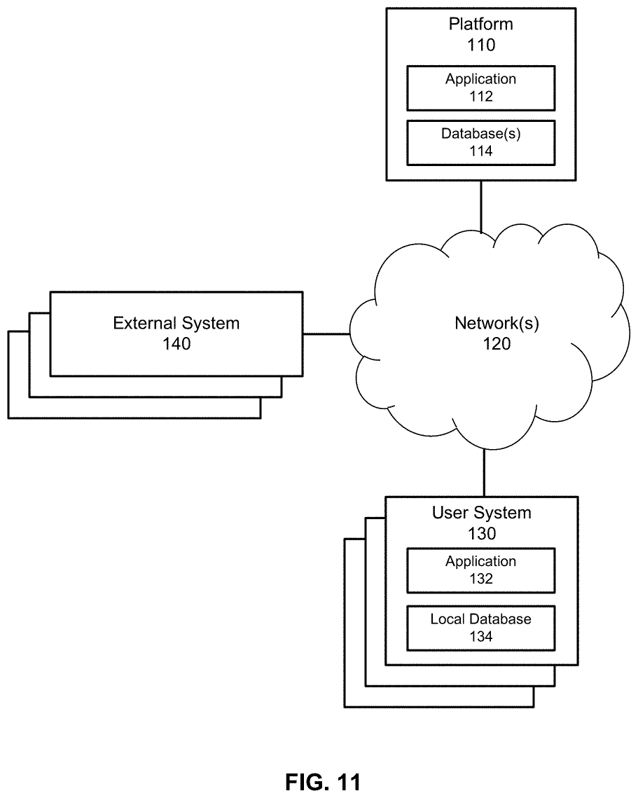

In addition to the access control and registration processes described above, device to device communication capability can also be used to track actual contacts. FIG. 2 is a diagram illustrating the installation of an application 202 onto a user system 204 9as described with respect to FIG. 11, such as a smartphone, tablet, wearable device, or other mobile user device and that can perform and manage the device to device communication in accordance with one embodiment. Application 202 can be installed over the air, upon manufacture of the device, or via a download initiated by the mobile device user.

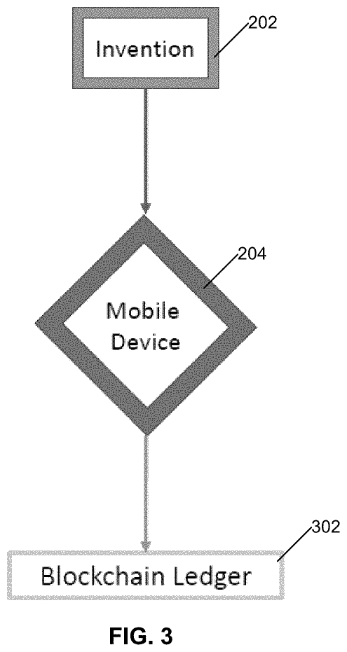

FIG. 3 is a diagram illustrating how the application 202 and user 204 of FIG. 2 can interface with a blockchain ledger 302 in order to securely maintain the contact information in accordance with one example embodiment. In accordance with FIG. 3, application 202 can cause user system 204 to communicate with the blockchain ledger 302 to obtain a Blockchain ID for the user system 204 upon or after Installation.

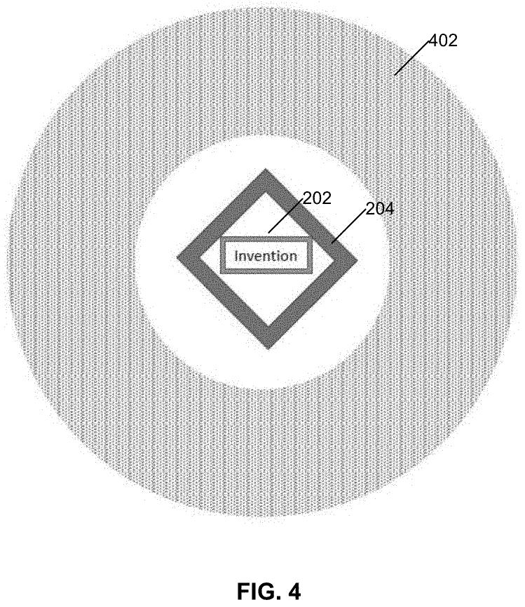

FIG. 4 is a diagram illustrating a ping radius for user system 204 initiated under the control of application 202 in accordance with one example embodiment. Once installed application 202 can cause user system 204 to “ping’ nearby user system with a certain pinging radius 402. Those of skill in the art will understand that a ping as used with respect to communications refers to a utility used to test the reachability of another device on a common network or using a common communication protocol.

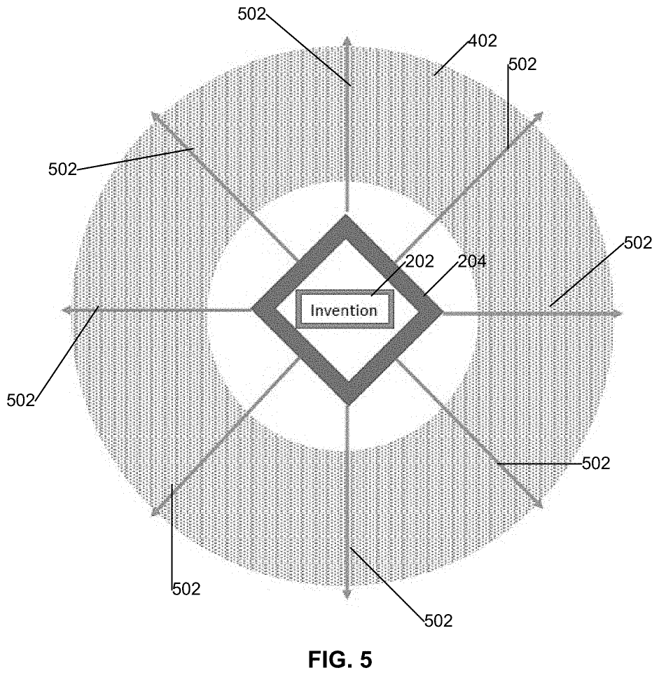

For example, application 202 can determine the available wireless technologies for user system 204. The application 202 can cause the user system 204, using one or more of the available wireless technologies, to issue pings 502 using a minimum or custom time interval, which defines the ping radius, as illustrated in FIG. 5. In certain embodiments, location information for user system 204 and surrounding devices 602 can be used to initiate pings 502.

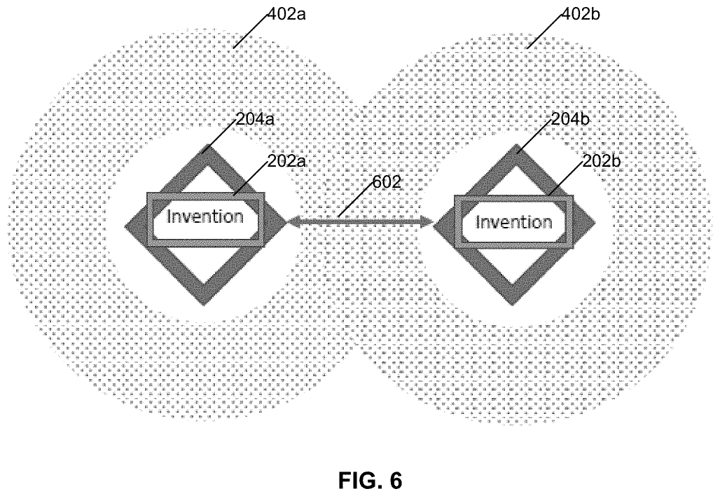

FIG. 6 illustrates that when an application 202a identifies a user system 202b within the ping radius 204a,b, then the application 202a can send contact data 602, or request contact data from application 202b. The contact data is extremely valuable for purposes of contact tracing as the contact data 602 can provide geolocation and physical proximity for the user systems that include the applications 202a,b.

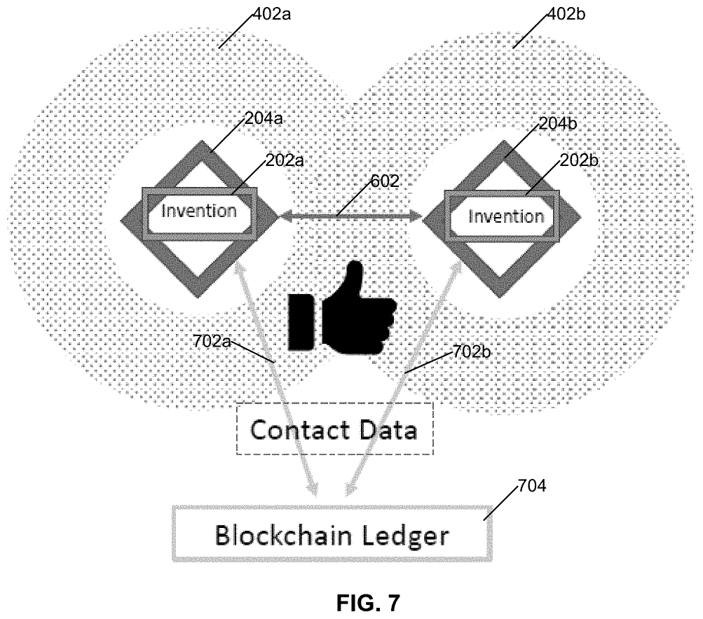

As illustrated in FIG. 7, the contact data 702a,b from, e.g., each of user system 204a,b can then be stored for future use in contact tracing. In certain embodiments, the contact data can be entered into a blockchain ledger 704 as a block chain contract for added security. This can then occur, for all contacts of both of devices 202a and 202b, and so on and so on. Thus, if the user of a device 202a or b is determined to be infected, then the contact data can be accessed and used to trace all of the user's contacts that may also be at risk of infection.

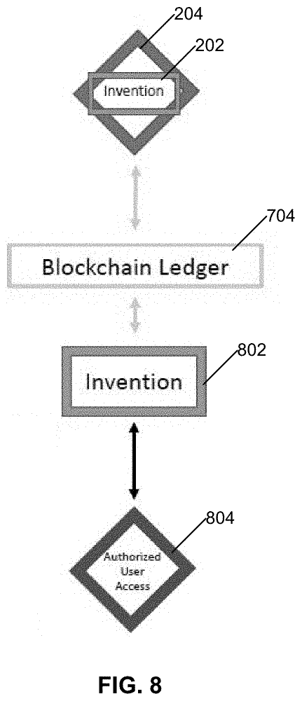

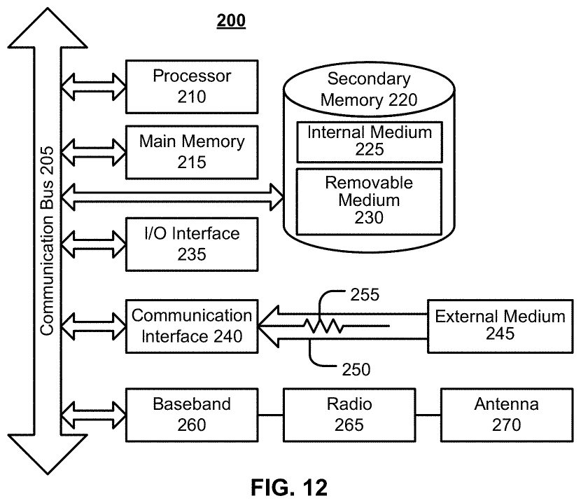

As illustrated in FIG. 8, an authorized user 804 can then access the contact data 702, e.g., in the blockchain ledger 704 through their own hardware, e.g., computer with the appropriate software 802, as described with respect to FIGS. 11 and 12. The authorized user 804 can have access to relevant data on the blockchain ledger 704, without compromising privacy laws or ethics.

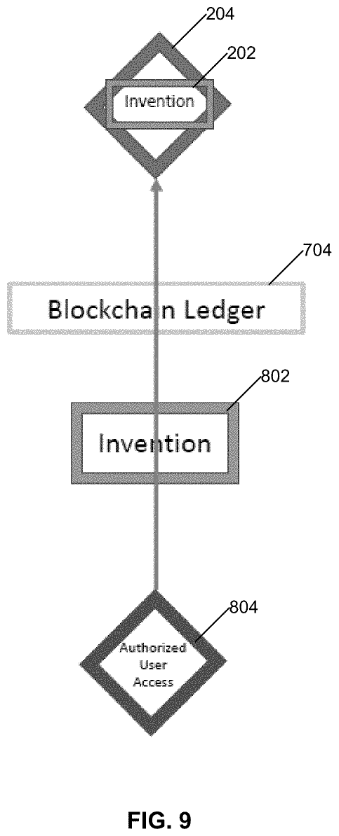

As illustrated in FIG. 9, when so empowered by, e.g., a government, the authorized user 804 can directly access the device 204, including the related contact data, geolocation (past/future probability), or even calling and texting individually or in mass. This information can then be used to follow up with those contacts, and their contacts, etc., using, e.g., the workflows and processed described below.

Thus, as described above, contact data can be obtain via “digital handshakes” or pings and corresponding exchange of contact data, text messages, websites/API forms, QR codes, etc. Another way to identify a close contact is where a possible patient under investigation is identified through other means than “digital handshakes” such as text messages, emails, interactive voice recordings, website input, or integration with other data systems. An example would be when a patient under investigation provides their close contacts to the health agency, which require the process to start over again to identify that person's close contacts, where such patient is to provide people who may constitute a close contact of that patient under investigation.

All of this contact data can be sent to a secure database(s) and/or blockchain ledger 704 to manage potential infection risk. The contact data can comprise the contact data and time as well as location information for the user systems 204. The contact duration should also be stored, as it is important for contact tracing. Authorized users 804 can then enter virus or contagion testing results and/or risk factors and associate them with an, e.g., block chain identification (ID). This can cause the information to then be securely associated with a user device 204 and creates a scalable resource for contact tracing.

An authorized user can now query, e.g., a blockchain ledger or other database to unanimously see contact data for a large plurality of users and initiate digital handshakes as necessary all anonymously, i.e., the authorized user does not need to know the identity of any of the users. If the authorized user 804 wants to see where a user was during a given time, e.g., a time period when they may have been exposed or exposed others to a virus, the authorized user can query the ledger or database and get GPS or other location data for the user during that period. In certain embodiments, the authorized user 804 can see a map the coordinates or location data. The authorized user can also see who the user came in contact with during that period. The authorized user can then start digital handshakes with those contacts and potentially with any businesses or other entities the user visited.

Health agencies can then view their local population, e.g., identified by numbers associated with PII/PHI, from, e.g., a PC dashboard in a CRM-style interface so they can manage infection risks by DEFAULT or by CUSTOM stratification. DEFAULT risk can be stratified based upon “degrees of separation” from a known or likely infection of a disease. For instance, patients under investigation from digital handshakes, or positive tests imported or integrated with the system, or any proxy for a positive test, i.e., information known to constitute a likely infection like a survey provided to a user of the system via app., chat, text, interactive voice recording, email, website, or form on a website, or that may be determined according to local protocols of a health agency, can be categorized by DEFAULT as “Positive” risk. Close contacts of a “Positive” risk are “High” risk.

Digital handshakes record “close contacts” and contact data related thereto can then be sent to a secure database, which for Covid-19, means when people or devices spend 15-minutes at 6-feet or less distance, for example. Close contacts are categorized by DEFAULT as “High” risk. Other contagions or diseases may have different parameters for what constitutes a “close contact.”

Close contacts of close contacts can be deemed secondary contacts, which can be categorized by DEFAULT as “Medium” risk. These are, e.g., for COVID-19, a person/device that has spent 15-minutes at 6-feet or less distance to a close contact.

Close contacts of secondary contacts can be deemed tertiary contacts, which can be categorized by DEFAULT as “Low” risk. These are, e.g., for COVID-19, a person/device that has spent 15-minutes at 6-feet or less distance to a secondary contact.

Any patient under investigation who is no longer in the queue due to a negative test or other parameters defined by the health agency or administration becomes “resolved” or “recovered.”

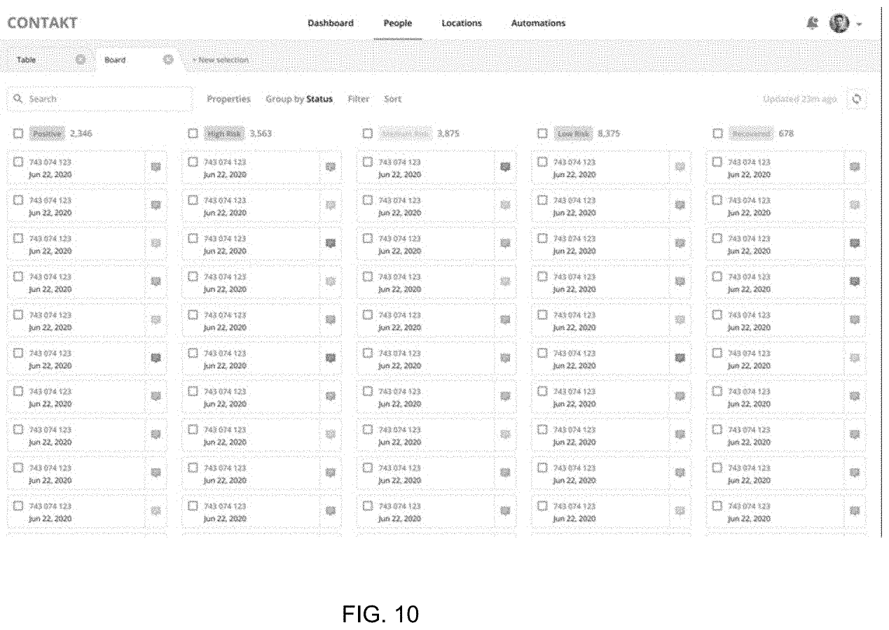

The dashboard illustrated in FIG. 10 is an example dashboard that can be used by an authorized user 804 to manage contact tracing according to the above DEFAULT classifications. As can be seen, the user data is anonymized and only the device identifier is available. The device identifier, in this case the device number can be used to send communications, e.g., texts, calls, emails, etc., to the users of the devices 204 in order to provide information and instructions and to get information related to their condition and symptoms, etc.

For patients under investigation starting from inbound text messages, emails, interactive voice recordings, websites, website forms/APIs, apps., or chat from online and offline marketing placed throughout the communities or in businesses, etc., positive tests imported or integrated with the system, or any proxy for a positive test, i.e. information known to constitute a likely infection like a survey provided to a user of the system via app., chat, text, interactive voice recording, email, website, or form on a website, however that may be determined according to local protocols of a health agency, are categorized by DEFAULT as “Positive” risk. Close contacts of a “Positive” risk are then deemed to be a “High” risk, etc.

CUSTOM risk can be stratified when either validated scientific data is received or additional risk parameters associated with close contacts and native or integrated testing results are learned to further enhance the data's accuracy over time through artificial intelligence or algorithms. These risk parameters, which can replace or supplement DEFAULT risk, include but are not limited to known cohabitants of a “Positive” risk, as measured by their GPS coordinates for durations, or the recurring digital handshakes that would suggest the same, case investigation that shows cohabitants or close relationships through external data sources, longer duration of exposure for a contagion, e.g. 50% more than 15-minutes that constitutes a close contact, closer proximity of exposure for a contagion, e.g. 1-feet instead of 6-feet, native or externally integrated survey data that would suggest repeated close contacts, native or externally integrated survey data that would suggest co-morbidities known to enhance risk of adverse outcome for a given contagion, Electronic Health Record (EHR) integration which would suggest co-morbidities known to enhance risk of adverse outcome for a given contagion, close contacts which occur indoors vs. outdoors, and vice versa, close contacts which occur in a high-risk environment outdoors or indoors, where a given contagion is more likely to be transmitted, and/or type of and manufacturer of a test conducted for a contagion, or frequency of testing, to account for the statistical relevance or accuracy of a given type of test and the likelihood of infection based on the results.

Demographic information based on other inputs like zip codes or data integrations can also be obtained and used by the platform as information for risk stratification.

Thus, all of the above contact information can be used to track a population and their contacts, identify and stratify risk, follow up with individuals and their contacts as appropriate given the associated risk, etc. In other words, e.g., a health agency can use information according to the risk of the people/devices recorded to a secure database. Such a health agency or other organization will likely want to reach its population/events to notify them of certain risks. Using the information present, e.g., via the dashboard of FIG. 10, a health agency or business/site can contact individuals or places via a call, text, sending an interactive voice recording, or an email that contains a message, survey, or instructions on what the recipient should do.

The data presented in the dashboard can be further anonymized such that the health agency or business/site will not even know the mobile number, landline number, or email address—they just have the ability to use the anonymized version to reach the individual/device.

The recipient of a follow up from the health agency or business can respond in multiple modes. They can use an app, they can use chat from an app or website or text, or they can respond via text or email, they can request a phone call, or they can request an interactive voice recording to respond to prompts.

The health agency or business can implement DEFAULT workflows provided via the user interface that includes the dashboard of FIG. 10. In other words, the platform can help the organization reach people through a defined process, e.g. a text, email, and/or IVR call every day until response is received, after which they get a text every day about how they are feeling or if they need anything, etc.

In certain embodiments, the health agency or business can define their own protocols, creating a CUSTOM workflow. This will be based on the organization's own local protocols, and can be added to the DEFAULT workflows. This may include directing close contacts to local testing, e.g., with a QR code. Or it could mean asking a local organization for a quarantine kit.

In certain embodiments, the organization and the recipient can determine the language that they prefer at any time, which will change the communications to suit that individual sender or recipient until it is changed again.

A health agency or business/site can automate certain protocols for risk levels. For instance, a health agency or business could automatically reach out to all HIGH risks immediately with surveys and directions to quarantine, followed by a prompt for the health agency to call them personally. Or, all Medium and Low risks could have an automated process, and a health agency only focuses their “manual” efforts and calls/texts/emails to the High risks and Positive cases.

The data gathered, e.g., for contact tracing, follow ups, interventions, etc., can be used to recommend intervention protocols to use by, e.g., health agencies. In other words, the data and workflows can be used to see how well workflows and interventions are working in order to make recommendations, and then health agencies and sites can select from the most effective.

FIG. 11 illustrates an example infrastructure in which one or more of the disclosed processes may be implemented, according to an embodiment. The infrastructure may comprise a platform 110 (e.g., one or more servers) which hosts and/or executes one or more of the various functions, processes, methods, and/or software modules described herein. Platform 110 may comprise dedicated servers, or may instead comprise cloud instances, which utilize shared resources of one or more servers. These servers or cloud instances may be collocated and/or geographically distributed. Platform 110 may also comprise or be communicatively connected to a server application 112 and/or one or more databases 114. In addition, platform 110 may be communicatively connected to one or more user systems 130 via one or more networks 120. Platform 110 may also be communicatively connected to one or more external systems 140 (e.g., other platforms, websites, etc.) via one or more networks 120.

Network(s) 120 may comprise the Internet, and platform 110 may communicate with user system(s) 130 through the Internet using standard transmission protocols, such as HyperText Transfer Protocol (HTTP), HTTP Secure (HTTPS), File Transfer Protocol (FTP), FTP Secure (FTPS), Secure Shell FTP (SFTP), and the like, as well as proprietary protocols. While platform 110 is illustrated as being connected to various systems through a single set of network(s) 120, it should be understood that platform 110 may be connected to the various systems via different sets of one or more networks. For example, platform 110 may be connected to a subset of user systems 130 and/or external systems 140 via the Internet, but may be connected to one or more other user systems 130 and/or external systems 140 via an intranet. Furthermore, while only a few user systems 130 and external systems 140, one server application 112, and one set of database(s) 114 are illustrated, it should be understood that the infrastructure may comprise any number of user systems, external systems, server applications, and databases.

User system(s) 130 may comprise any type or types of computing devices capable of wired and/or wireless communication, including without limitation, desktop computers, laptop computers, tablet computers, smart phones or other mobile phones, servers, game consoles, televisions, set-top boxes, electronic kiosks, point-of-sale terminals, Automated Teller Machines, and/or the like.

Platform 110 may comprise web servers which host one or more websites and/or web services. In embodiments in which a website is provided, the website may comprise a graphical user interface, including, for example, one or more screens (e.g., webpages) generated in HyperText Markup Language (HTML) or other language. Platform 110 transmits or serves one or more screens of the graphical user interface in response to requests from user system(s) 130. In some embodiments, these screens may be served in the form of a wizard, in which case two or more screens may be served in a sequential manner, and one or more of the sequential screens may depend on an interaction of the user or user system 130 with one or more preceding screens. The requests to platform 110 and the responses from platform 110, including the screens of the graphical user interface, may both be communicated through network(s) 120, which may include the Internet, using standard communication protocols (e.g., HTTP, HTTPS, etc.). These screens (e.g., webpages) may comprise a combination of content and elements, such as text, images, videos, animations, references (e.g., hyperlinks), frames, inputs (e.g., textboxes, text areas, checkboxes, radio buttons, drop-down menus, buttons, forms, etc.), scripts (e.g., JavaScript), and the like, including elements comprising or derived from data stored in one or more databases (e.g., database(s) 114) that are locally and/or remotely accessible to platform 110. Platform 110 may also respond to other requests from user system(s) 130.

Platform 110 may further comprise, be communicatively coupled with, or otherwise have access to one or more database(s) 114. For example, platform 110 may comprise one or more database servers which manage one or more databases 114. A user system 130 or server application 112 executing on platform 110 may submit data (e.g., user data, form data, etc.) to be stored in database(s) 114, and/or request access to data stored in database(s) 114. Any suitable database may be utilized, including without limitation My SQL™, Oracle™ IBM™, Microsoft SQL™, Access™, PostgreSQL™, and the like, including cloud-based databases and proprietary databases. Data may be sent to platform 110, for instance, using the well-known POST request supported by HTTP, via FTP, and/or the like. This data, as well as other requests, may be handled, for example, by server-side web technology, such as a servlet or other software module (e.g., comprised in server application 112), executed by platform 110.

In embodiments in which a web service is provided, platform 110 may receive requests from external system(s) 140, and provide responses in eXtensible Markup Language (XML), JavaScript Object Notation (JSON), and/or any other suitable or desired format. In such embodiments, platform 110 may provide an application programming interface (API) which defines the manner in which user system(s) 130 and/or external system(s) 140 may interact with the web service. Thus, user system(s) 130 and/or external system(s) 140 (which may themselves be servers), can define their own user interfaces, and rely on the web service to implement or otherwise provide the backend processes, methods, functionality, storage, and/or the like, described herein. For example, in such an embodiment, a client application 132 executing on one or more user system(s) 130 may interact with a server application 112 executing on platform 110 to execute one or more or a portion of one or more of the various functions, processes, methods, and/or software modules described herein. Client application 132 may be “thin,” in which case processing is primarily carried out server-side by server application 112 on platform 110. A basic example of a thin client application 132 is a browser application, which simply requests, receives, and renders webpages at user system(s) 130, while server application 112 on platform 110 is responsible for generating the webpages and managing database functions. Alternatively, the client application may be “thick,” in which case processing is primarily carried out client-side by user system(s) 130. It should be understood that client application 132 may perform an amount of processing, relative to server application 112 on platform 110, at any point along this spectrum between “thin” and “thick,” depending on the design goals of the particular implementation. In any case, the application described herein, which may wholly reside on either platform 110 (e.g., in which case server application 112 performs all processing) or user system(s) 130 (e.g., in which case client application 132 performs all processing) or be distributed between platform 110 and user system(s) 130 (e.g., in which case server application 112 and client application 132 both perform processing), can comprise one or more executable software modules that implement one or more of the processes, methods, or functions of the application described herein.

FIG. 12 is a block diagram illustrating an example wired or wireless system 550 that can be used in connection with various embodiments described herein. For example, the system 550 can be used as or in conjunction with one or more of the platforms, devices or processes described above, and may represent components of the mobile devices, the corresponding backend server(s), and/or other devices described herein. The system 550 can be a server or any conventional personal computer, or any other processor-enabled device that is capable of wired or wireless data communication. Other computer systems and/or architectures may be also used, as will be clear to those skilled in the art.

The system 550 preferably includes one or more processors, such as processor 560. Additional processors may be provided, such as an auxiliary processor to manage input/output, an auxiliary processor to perform floating point mathematical operations, a special-purpose microprocessor having an architecture suitable for fast execution of signal processing algorithms (e.g., digital signal processor), a slave processor subordinate to the main processing system (e.g., backend processor), an additional microprocessor or controller for dual or multiple processor systems, or a coprocessor. Such auxiliary processors may be discrete processors or may be integrated with the processor 560. Examples of processors which may be used with system 550 include, without limitation, the Pentium® processor, Core i7® processor, and Xeon® processor, all of which are available from Intel Corporation of Santa Clara, Calif.

The processor 560 is preferably connected to a communication bus 555. The communication bus 555 may include a data channel for facilitating information transfer between storage and other peripheral components of the system 550. The communication bus 555 further may provide a set of signals used for communication with the processor 560, including a data bus, address bus, and control bus (not shown). The communication bus 555 may comprise any standard or non-standard bus architecture such as, for example, bus architectures compliant with industry standard architecture (ISA), extended industry standard architecture (EISA), Micro Channel Architecture (MCA), peripheral component interconnect (PCI) local bus, or standards promulgated by the Institute of Electrical and Electronics Engineers (IEEE) including IEEE 488 general-purpose interface bus (GPM), IEEE 696/S-100, and the like.

System 550 preferably includes a main memory 565 and may also include a secondary memory 570. The main memory 565 provides storage of instructions and data for programs executing on the processor 560, such as one or more of the functions and/or modules discussed above. It should be understood that programs stored in the memory and executed by processor 560 may be written and/or compiled according to any suitable language, including without limitation C/C++, Java, JavaScript, Pearl, Visual Basic, .NET, and the like. The main memory 565 is typically semiconductor-based memory such as dynamic random access memory (DRAM) and/or static random access memory (SRAM). Other semiconductor-based memory types include, for example, synchronous dynamic random access memory (SDRAM), Rambus dynamic random access memory (RDRAM), ferroelectric random access memory (FRAM), and the like, including read only memory (ROM).

The secondary memory 570 may optionally include an internal memory 575 and/or a removable medium 580, for example a floppy disk drive, a magnetic tape drive, a compact disc (CD) drive, a digital versatile disc (DVD) drive, other optical drive, a flash memory drive, etc. The removable medium 580 is read from and/or written to in a well-known manner. Removable storage medium 580 may be, for example, a floppy disk, magnetic tape, CD, DVD, SD card, etc.

The removable storage medium 580 is a non-transitory computer-readable medium having stored thereon computer executable code (i.e., software) and/or data. The computer software or data stored on the removable storage medium 580 is read into the system 550 for execution by the processor 560.

In alternative embodiments, secondary memory 570 may include other similar means for allowing computer programs or other data or instructions to be loaded into the system 550. Such means may include, for example, an external storage medium 595 and an interface 590. Examples of external storage medium 595 may include an external hard disk drive or an external optical drive, or and external magneto-optical drive.

Other examples of secondary memory 570 may include semiconductor-based memory such as programmable read-only memory (PROM), erasable programmable read-only memory (EPROM), electrically erasable read-only memory (EEPROM), or flash memory (block oriented memory similar to EEPROM). Also included are any other removable storage media 580 and communication interface 590, which allow software and data to be transferred from an external medium 595 to the system 550.

System 550 may include a communication interface 590. The communication interface 590 allows software and data to be transferred between system 550 and external devices (e.g. printers), networks, or information sources. For example, computer software or executable code may be transferred to system 550 from a network server via communication interface 590. Examples of communication interface 590 include a built-in network adapter, network interface card (NIC), Personal Computer Memory Card International Association (PCMCIA) network card, card bus network adapter, wireless network adapter, Universal Serial Bus (USB) network adapter, modem, a network interface card (NIC), a wireless data card, a communications port, an infrared interface, an IEEE 1394 fire-wire, or any other device capable of interfacing system 550 with a network or another computing device.

Communication interface 590 preferably implements industry promulgated protocol standards, such as Ethernet IEEE 802 standards, Fiber Channel, digital subscriber line (DSL), asynchronous digital subscriber line (ADSL), frame relay, asynchronous transfer mode (ATM), integrated digital services network (ISDN), personal communications services (PCS), transmission control protocol/Internet protocol (TCP/IP), serial line Internet protocol/point to point protocol (SLIP/PPP), and so on, but may also implement customized or non-standard interface protocols as well.

Software and data transferred via communication interface 590 are generally in the form of electrical communication signals 605. These signals 605 are preferably provided to communication interface 590 via a communication channel 600. In one embodiment, the communication channel 600 may be a wired or wireless network, or any variety of other communication links. Communication channel 600 carries signals 605 and can be implemented using a variety of wired or wireless communication means including wire or cable, fiber optics, conventional phone line, cellular phone link, wireless data communication link, radio frequency (“RF”) link, or infrared link, just to name a few.

Computer executable code (i.e., computer programs or software) is stored in the main memory 565 and/or the secondary memory 570. Computer programs can also be received via communication interface 590 and stored in the main memory 565 and/or the secondary memory 570. Such computer programs, when executed, enable the system 550 to perform the various functions of the present invention as previously described.

In this description, the term “computer readable medium” is used to refer to any non-transitory computer readable storage media used to provide computer executable code (e.g., software and computer programs) to the system 550. Examples of these media include main memory 565, secondary memory 570 (including internal memory 575, removable medium 580, and external storage medium 595), and any peripheral device communicatively coupled with communication interface 590 (including a network information server or other network device). These non-transitory computer readable mediums are means for providing executable code, programming instructions, and software to the system 550.

In an embodiment that is implemented using software, the software may be stored on a computer readable medium and loaded into the system 550 by way of removable medium 580, I/O interface 585, or communication interface 590. In such an embodiment, the software is loaded into the system 550 in the form of electrical communication signals 605. The software, when executed by the processor 560, preferably causes the processor 560 to perform the inventive features and functions previously described herein.

In an embodiment, I/O interface 585 provides an interface between one or more components of system 550 and one or more input and/or output devices. Example input devices include, without limitation, keyboards, touch screens or other touch-sensitive devices, biometric sensing devices, computer mice, trackballs, pen-based pointing devices, and the like. Examples of output devices include, without limitation, cathode ray tubes (CRTs), plasma displays, light-emitting diode (LED) displays, liquid crystal displays (LCDs), printers, vacuum florescent displays (VFDs), surface-conduction electron-emitter displays (SEDs), field emission displays (FEDs), and the like.

The system 550 also includes optional wireless communication components that facilitate wireless communication over a voice and over a data network. The wireless communication components comprise an antenna system 610, a radio system 615 and a baseband system 620. In the system 550, radio frequency (RF) signals are transmitted and received over the air by the antenna system 610 under the management of the radio system 615.

In one embodiment, the antenna system 610 may comprise one or more antennae and one or more multiplexors (not shown) that perform a switching function to provide the antenna system 610 with transmit and receive signal paths. In the receive path, received RF signals can be coupled from a multiplexor to a low noise amplifier (not shown) that amplifies the received RF signal and sends the amplified signal to the radio system 615.

In alternative embodiments, the radio system 615 may comprise one or more radios that are configured to communicate over various frequencies. In one embodiment, the radio system 615 may combine a demodulator (not shown) and modulator (not shown) in one integrated circuit (IC). The demodulator and modulator can also be separate components. In the incoming path, the demodulator strips away the RF carrier signal leaving a baseband receive audio signal, which is sent from the radio system 615 to the baseband system 620.

If the received signal contains audio information, then baseband system 620 decodes the signal and converts it to an analog signal. Then the signal is amplified and sent to a speaker. The baseband system 620 also receives analog audio signals from a microphone. These analog audio signals are converted to digital signals and encoded by the baseband system 620. The baseband system 620 also codes the digital signals for transmission and generates a baseband transmit audio signal that is routed to the modulator portion of the radio system 615. The modulator mixes the baseband transmit audio signal with an RF carrier signal generating an RF transmit signal that is routed to the antenna system and may pass through a power amplifier (not shown). The power amplifier amplifies the RF transmit signal and routes it to the antenna system 610 where the signal is switched to the antenna port for transmission.

The baseband system 620 is also communicatively coupled with the processor 560. The central processing unit 560 has access to data storage areas 565 and 570. The central processing unit 560 is preferably configured to execute instructions (i.e., computer programs or software) that can be stored in the memory 565 or the secondary memory 570. Computer programs can also be received from the baseband processor 610 and stored in the data storage area 565 or in secondary memory 570, or executed upon receipt. Such computer programs, when executed, enable the system 550 to perform the various functions of the present invention as previously described. For example, data storage areas 565 may include various software modules (not shown).

Various embodiments may also be implemented primarily in hardware using, for example, components such as application specific integrated circuits (ASICs), or field programmable gate arrays (FPGAs). Implementation of a hardware state machine capable of performing the functions described herein will also be apparent to those skilled in the relevant art. Various embodiments may also be implemented using a combination of both hardware and software.

Furthermore, those of skill in the art will appreciate that the various illustrative logical blocks, modules, circuits, and method steps described in connection with the above described figures and the embodiments disclosed herein can often be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a module, block, circuit or step is for ease of description. Specific functions or steps can be moved from one module, block or circuit to another without departing from the invention.

Moreover, the various illustrative logical blocks, modules, functions, and methods described in connection with the embodiments disclosed herein can be implemented or performed with a general purpose processor, a digital signal processor (DSP), an ASIC, FPGA or other programmable logic device, discrete gate or transistor logic, discrete hardware components, or any combination thereof designed to perform the functions described herein. A general-purpose processor can be a microprocessor, but in the alternative, the processor can be any processor, controller, microcontroller, or state machine. A processor can also be implemented as a combination of computing devices, for example, a combination of a DSP and a microprocessor, a plurality of microprocessors, one or more microprocessors in conjunction with a DSP core, or any other such configuration.

Additionally, the steps of a method or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of storage medium including a network storage medium. An exemplary storage medium can be coupled to the processor such the processor can read information from, and write information to, the storage medium. In the alternative, the storage medium can be integral to the processor. The processor and the storage medium can also reside in an ASIC.

Any of the software components described herein may take a variety of forms. For example, a component may be a stand-alone software package, or it may be a software package incorporated as a “tool” in a larger software product. It may be downloadable from a network, for example, a website, as a stand-alone product or as an add-in package for installation in an existing software application. It may also be available as a client-server software application, as a web-enabled software application, and/or as a mobile application.

While certain embodiments have been described above, it will be understood that the embodiments described are by way of example only. Accordingly, the systems and methods described herein should not be limited based on the described embodiments. Rather, the systems and methods described herein should only be limited in light of the claims that follow when taken in conjunction with the above description and accompanying drawings.

What is claimed:

1. A user system comprising:
at least one hardware processor; and
one or more software modules that are configured to, when executed by the at least one hardware processor,
download an application to the user system;
obtain an identifier for the user system;
generate a plurality of pings to detect other nearby user systems;
receive a response to one of the plurality of pings from another user system;
exchange contact data with the responding user system;
store the contact data for the responding user system; and
receive a digital handshake indicated, based on the stored contact data that a user of the user system may be at risk.

2. The user system of claim 1, further comprising wireless communication capability, and wherein the one or more software modules are further configured to, when executed by the at least one hardware processor, detect the wireless communication capability and generate the pings using the detecting wireless capability.

3. The user system of claim 1, wherein the one or more software modules are further configured to, when executed by the at least one hardware processor, generate the pings using a time interval that defines a ping radius.

4. The user system of claim 1, wherein the contact data comprises at least on of an identifier of the responding user system, location information, proximity information and contact duration.

5. The user system of claim 1, wherein the contact data comprises at least on of an identifier of the responding user system, location information, proximity information or contact duration.

6. The user system of claim 1, wherein the identification is a blockchain identification and the contact data is stored as a blockchain contract in a blockchain ledger.

7. A system comprising:
at least one hardware processor; and
one or more software modules that are configured to, when executed by the at least one hardware processor,
download an application to each of a plurality of user systems;
record an identification for each of the plurality of user systems;
receive contact data for each of the plurality of user systems;
store the contact data for a given on of the plurality of user systems in a record of a plurality of records, wherein the record is associated with the related identifier;
receive additional information from an authorized user related to one or more of the plurality of records; and
initiate a digital handshake with at least one of the plurality of user devices based on the contact data and the additional data.

8. The system of claim 7, wherein the one or more software modules are further configured to, when executed by the at least one hardware processor, receive queries related to the plurality of records and return anonymous contact data in response to the queries.

9. The user system of claim 7, wherein the contact data comprises at least on of an identifier of the corresponding user system, location information, proximity information and contact duration.

10. The user system of claim 7, wherein the contact data comprises at least on of an identifier of the responding user system, location information, proximity information or contact duration.

11. The user system of claim 7, wherein the identifications for each of the plurality of user systems is a blockchain identification and each of the plurality of records is a blockchain contract in a blockchain ledger.

12. A method comprising:
downloading an application to a user system;
obtaining an identifier for the user system;
generating a plurality of pings to detect other nearby user systems;
receiving a response to one of the plurality of pings from another user system;
exchanging contact data with the responding user system;
storing the contact data for the responding user system; and
receiving a digital handshake indicated, based on the stored contact data that a user of the user system may be at risk.

13. The method of claim 12, further comprising detecting the wireless communication capability and generating the pings using the detecting wireless capability.

14. The method of claim 12, further comprising generating the pings using a time interval that defines a ping radius.

15. The user system of claim 1, wherein the contact data comprises at least on of an identifier of the responding user system, location information, proximity information and contact duration.

16. The method of claim 12, wherein the contact data comprises at least on of an identifier of the responding user system, location information, proximity information or contact duration.

17. The method of claim 12, wherein the identification is a blockchain identification and the contact data is stored as a blockchain contract in a blockchain ledger.

18. A method comprising:
downloading an application to each of a plurality of user systems;
recording an identification for each of the plurality of user systems;
receiving contact data for each of the plurality of user systems;
storing the contact data for a given on of the plurality of user systems in a record of a plurality of records, wherein the record is associated with the related identifier;
receiving additional information from an authorized user related to one or more of the plurality of records; and
initiating a digital handshake with at least one of the plurality of user devices based on the contact data and the additional data.

19. The method of claim 18, further comprising receiving queries related to the plurality of records and returning anonymous contact data in response to the queries.

20. The method of claim 18, wherein the contact data comprises at least on of an identifier of the corresponding user system, location information, proximity information and contact duration.

21. The method of claim 18, wherein the contact data comprises at least on of an identifier of the responding user system, location information, proximity information or contact duration.

22. The method of claim 18, wherein the identifications for each of the plurality of user systems is a blockchain identification and each of the plurality of records is a blockchain contract in a blockchain ledger.
